(19) United States
(12) Patent Application Publication
(10) Pub. No.: US 2023/0398367 A1
(43) Pub. Date: Dec. 14, 2023

(54) SYSTEMS AND METHODS FOR INCREASING INTESTINAL ABSORPTION OF THERAPEUTIC AGENTS

(71) Applicant: Novocure GmbH, Root D4 (CH)

(72) Inventors: Moshe Giladi, Haifa (IL); Tali Voloshin-Sela, Haifa (IL); Lilach Avigdor, Haifa (IL); Gadi Cohen, Haifa (IL)

(73) Assignee: Novocure GmbH, Root D4 (CH)

(21) Appl. No.: 18/333,906

(22) Filed: Jun. 13, 2023

Related U.S. Application Data
(60) Provisional application No. 63/351,561, filed on Jun. 13, 2022.

(51) Int. Cl. A61N 1/40

(52) U.S. Cl. CPC A61N 1/40

(57) ABSTRACT

Systems and methods for increasing absorption of ingested substances into a subject's bloodstream are disclosed. The systems and methods involve application of an alternating field to the subject's abdomen, and may further include oral administration of at least one substance to the subject. Also disclosed are methods of treating or reducing the occurrence of a malabsorption condition in a subject via application of the alternating electric field to the subject's abdomen.

### CROSS REFERENCE TO RELATED APPLICATIONS/INCORPORATION BY REFERENCE STATEMENT

The subject application claims benefit under 35 USC § 119(e) of U.S. provisional application No. 63/351,561, filed Jun. 13, 2022. The entire contents of the above-referenced patent application(s) are hereby expressly incorporated herein by reference.

### STATEMENT REGARDING FEDERALLY SPONSORED RESEARCH OR DEVELOPMENT

Not Applicable.

### BACKGROUND

Oral administration is one of the most common administration routes utilized for delivery of therapeutic substances. Absorption of drugs is mostly done in the small intestine, since it has the largest surface area; around 60% of drug emptied from the stomach is absorbed from the duodenum (Jobin, et al. (1985) Br. J. Clin. Pharmac., 19:97S-105S; Hua S (2020) Front. Pharmacol., 11:524). However, various physiological barriers exist that limit the amount of ingested substance that is absorbed through the intestine and into the bloodstream.

Claudin-5 is expressed in the duodenum and colon, which have the strongest expression of “tightening” from the group of cation barriers that are claudin-1, -3, -4, and -5 in the colon (Garcia-Hernandez, et al. (2017 June) Ann N Y Acad Sci., 1397 (1):66-79). Dysregulation of claudin proteins has been observed in inflammatory states associated with a leaky epithelial barrier. While abundant claudin-5 expression was identified in the duodenum, peak claudin-2, -5, -7, and -10 mRNA expression has been noted in tight junctions (TJs) of epithelial cells in the ileocecal junction, and downregulation of claudin-3, -4, and -7 was reported in ulcerative colitis. In Crohn's disease, upregulation of claudin-1 and claudin-2 and decreased expression of intestinal epithelial claudin-3, -5 , and -8 have been observed (Lu, et al. (2013) Tissue Barriers, 1:e24978).

Modulation of tight junction permeability has often been attempted as a means for peptides or nanoparticles to cross the epithelium. To this end, two types of strategies have been adopted: a seemingly less controlled tight junction modulation, creating leaks that allow the passage of large molecules; and a more controlled tight junction modulation. In the former case, surface active so-called absorption enhancers have been used. Familiar examples are medium chain fatty acids and their derivatives. While these agents cause reversible leaks in tight junctions within narrow concentration intervals in vitro, these levels are difficult to maintain after oral or local administration in vivo (Lindmark, et al. (1995) J Pharmacol Exp Ther., 275 (2):958-64; Lindmark, et al. (1997) Pharm Res, 14 (7):930-5; and Twarog, et al. (2021) E J Pharmac Sci., 158:105685).

At higher concentrations, many of the surface active enhancers also interact with cell membranes, causing irreversible damage to the intestinal epithelium. Notably, the less surface active shorter medium chain fatty acid sodium caprylic acid (C8) is added in an oral formulation of the macrocyclic peptide drug Octreotide, which is under NDA review by the US FDA (Aguirre, et al. (2016) Advanced Drug Delivery Reviews, 106 (B): 223-241). Other examples of therapeutic formulations containing absorption enhancers that are under review include TPE®, Eligen®, Peptelligence™, and Uruguanlyin analogue® (all in Phase III trials); GIPET®, POD™, SmPill®, IN-105, Axess™, and HDV-1 (all in Phase II); NOD and Oshadi Icp (in Phase I); and TrabiOral™, Intravail®, Robotic pill, and Nanomega nanoparticles (in Preclinical) (see, for example, Lindmark, et al. (1995) J Pharmacol Exp Ther., 275 (2):958-64; Lindmark, et al. (1997) Pharm Res, 14 (7):930-5; Twarog, et al. (2021) European Journal of Pharmaceutical Sciences, 158:105685).

Since MLC phosphorylation is dynamically regulated in polarized epithelial cells by a specific kinase (MLCK) and a specific phosphatase (MLCP), methods to selectively block MLCP have been tested in regards to increasing absorption function as a means to transiently increase MLC phosphorylation by basal MLCK activity (Taverner, et al. (2015) J Controlled Release, 210:189-197). Several MLCP inhibitory peptides were developed. In untreated animals, fluorescently labeled insulin could be visualized along the epithelial brush border, and after treatment with MLCP inhibitory peptides, the labeled insulin could be seen penetrating between enterocytes and into the sub epithelial tissue (Lundquist, et al. (2016) Adv Drug Delivery Rev, 106:256-276).

Tumor Treating Fields (TTFields) are low intensity (e.g., 1-3 V/cm) alternating electric fields within the intermediate frequency range (100-500 kHz) that target solid tumors by disrupting mitosis. This non-invasive treatment targets solid tumors and is described, for example, in U.S. Pat. Nos. 7,016,725; 7,089,054; 7,333,852; 7,565,205; 8,244,345; 8,715,203; 8,764,675; 10,188,851; and 10,441,776. TTFields are typically delivered through two pairs of transducer arrays that generate perpendicular fields within the treated tumor; the electrode arrays that make up each of these pairs are positioned on opposite sides of the body part that is being treated. More specifically, for the OPTUNE® system, one pair of electrodes is located to the left and right (LR) of the tumor, and the other pair of electrodes is located anterior and posterior (AP) to the tumor. TTFields are approved for the treatment of glioblastoma multiforme (GBM), and may be delivered, for example, via the OPTUNE® system (Novocure Limited, St. Helier, Jersey), which includes transducer arrays placed on the patient's shaved head.

Each transducer array used for the delivery of TTFields in the OPTUNE® device comprises a set of ceramic disk electrodes, which are coupled to the patient's skin (such as, but not limited to, the patient's shaved head for treatment of GBM) through a layer of conductive medical gel. The purpose of the medical gel is to deform to match the body's contours and to provide good electrical contact between the arrays and the skin; as such, the gel interface bridges the skin and reduces interference. The device is intended to be continuously worn by the patient for 2-4 days before removal for hygienic care and re-shaving (if necessary), followed by reapplication with a new set of arrays. As such, the medical gel remains in substantially continuous contact with an area of the patient's skin for a period of 2-4 days at a time, and there is only a brief period of time in which the area of skin is uncovered and exposed to the environment before more medical gel is applied thereto.

Patent Application Publication No. US 2020/0061360 discloses that treatment with alternating electric fields increased permeability of the blood brain barrier (BBB) by reducing claudin-5 expression and therefore weakening epithelial tight junctions (TJs). While the BBB is a highly regulated and complex barrier of the brain that prevents most small molecules from crossing the BBB, the '630 application demonstrates that application of alternating electric fields treatment increased permeability of the BBB both in vitro and in vivo, thereby allowing molecules of at least 4 kDa to pass the BBB after treatment with alternating electric fields.

However, there is a need in the art for new and improved systems and methods for increasing absorption of ingested substances into a patient's bloodstream, as well as methods of treating various malabsorption conditions. It is to such new and improved systems and methods that the present disclosure is directed.

### BRIEF DESCRIPTION OF THE DRAWINGS

FIG. 1 graphically illustrates claudin-2 staining of colon and duodenum samples taken from healthy male and female rats treated with TTFields via a torso array in a frequency of 150 kHz for two weeks.

### DETAILED DESCRIPTION

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed inventive concept(s) shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All patents, published patent applications, and non-patent publications mentioned in the specification are indicative of the level of skill of those skilled in the art to which this presently disclosed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions, assemblies, systems, kits, and/or methods disclosed herein can be made and executed without undue experimentation in light of the present disclosure. While the compositions, assemblies, systems, kits, and methods of the inventive concept(s) have been described in terms of particular embodiments, it will be apparent to those of skill in the art that variations may be applied to the compositions and/or methods and in the steps or in the sequence of steps of the methods described herein without departing from the concept, spirit, and scope of the inventive concept(s). All such similar substitutions and modifications apparent to those skilled in the art are deemed to be within the spirit, scope, and concept of the inventive concept(s) as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (e.g., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. For example, the term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

The term “pharmaceutically acceptable” refers to compounds and compositions which are suitable for administration to humans and/or animals without undue adverse side effects such as (but not limited to) toxicity, irritation, and/or allergic response commensurate with a reasonable benefit/risk ratio.

The term “patient” or “subject” as used herein includes human and veterinary subjects. “Mammal” for purposes of treatment refers to any animal classified as a mammal, including (but not limited to) humans, domestic and farm animals, nonhuman primates, and any other animal that has mammary tissue.

The term “treatment” refers to both therapeutic treatment and prophylactic or preventative measures. Those in need of treatment include, but are not limited to, individuals already having a particular condition/disease/infection as well as individuals who are at risk of acquiring a particular condition/disease/infection (i.e., those needing prophylactic/preventative measures). The term “treating” refers to administering an agent/element/method to a patient for therapeutic and/or prophylactic/preventative purposes.

The term “therapeutic composition” or “pharmaceutical composition” as used herein refers to an agent that may be administered in vivo to bring about a therapeutic and/or prophylactic/preventative effect.

Administering a therapeutically effective amount or prophylactically effective amount is intended to provide a therapeutic benefit in the treatment, prevention, and/or management of a disease, condition, and/or infection. The specific amount that is therapeutically effective can be readily determined by the ordinary medical practitioner, and can vary depending on factors known in the art, such as (but not limited to) the type of condition/disease/infection, the patient's history and age, the stage of the condition/disease/infection, and the co-administration of other agents.

The term “effective amount” refers to an amount of a biologically active molecule or conjugate or derivative thereof, or an amount of a treatment protocol (e.g., an alternating electric field), sufficient to exhibit a detectable therapeutic effect without undue adverse side effects (such as (but not limited to) toxicity, irritation, and allergic response) commensurate with a reasonable benefit/risk ratio when used in the manner of the inventive concept(s). The therapeutic effect may include, for example but not by way of limitation, preventing, inhibiting, or reducing the occurrence of at least one condition, disease, and/or infection. The effective amount for a subject will depend upon the type of subject, the subject's size and health, the nature and severity of the condition/disease/infection to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

As used herein, the term “concurrent therapy” is used interchangeably with the terms “combination therapy” and “adjunct therapy,” and will be understood to mean that the patient in need of treatment is treated with or given another drug for the condition/disease/infection in conjunction with the treatments of the present disclosure. This concurrent therapy can be sequential therapy, where the patient is treated first with one treatment protocol/pharmaceutical composition and then the other treatment protocol/pharmaceutical composition, or the two treatment protocols/pharmaceutical compositions are given simultaneously.

Turning now to the inventive concept(s), certain non-limiting embodiments thereof include a method of increasing absorption of at least one ingested substance into a bloodstream of a subject. The method includes the steps of: applying an alternating electric field to at least a portion of an abdomen of the subject for a period of time, and orally administering the at least one substance to the subject. The method steps can be performed in any order and either simultaneously or wholly or partially sequentially. Application of the alternating electric field increases permeability of at least a portion of an intestinal epithelia of the subject, and the increased permeability of the intestinal epithelia allows the at least one substance to cross an intestinal wall into the bloodstream at an increased rate compared to the amount of substance that crosses the intestinal wall in the absence of the alternating electric field.

Any type of conductive or non-conductive electrode(s) and/or transducer array(s) that can be utilized for generating an alternating electric field that are known in the art or otherwise contemplated herein may be applied to the subject's abdomen for generation of the alternating electric field in accordance with the present disclosure. Non-limiting examples of electrodes and transducer arrays that can be utilized for generating an alternating electric field in accordance with the present disclosure include those that function as part of a TTFields system as described, for example but not by way of limitation, in U.S. Pat. Nos. 7,016,725; 7,089,054; 7,333,852; 7,565,205; 8,244,345; 8,715,203; 8,764,675; 10,188,851; 10,441,776; and 11,452,863; and in U.S. Patent Application Nos. US 2018/0001078; US 2018/0160933; US 2019/0117956; US 2019/0307781; and US 2019/0308016.

The alternating electric field may be generated at any frequency that increases permeability of at least a portion of the intestinal epithelia of the subject (such as, but not limited to, by loosening tight junctions between intestinal epithelial cells, delocalizing claudin-2 in intestinal tight junctions, etc.), in accordance with the present disclosure. For example (but not by way of limitation), the alternating electric field may have a frequency of about 50 kHz, about 60 kHz, about 70 kHz, about 75 kHz, about 80 kHz, about 90 kHz, about 100 kHz, about 110 kHz, about 120 kHz, about 125 kHz, about 130 kHz, about 140 kHz, about 150 kHz, about 175 kHz, about 200 kHz, about 225 kHz, about 250 kHz, about 275 kHz, about 300 kHz, about 325 kHz, about 350 kHz, about 375 kHz, about 400 kHz, about 425 kHz, about 450 kHz, about 475 kHz, about 500 kHz, about 550 kHz, about 600 kHz, about 650 kHz, about 700 kHz, about 750 kHz, about 800 kHz, about 850 kHz, about 900 kHz, about 950 kHz, about 1 MHz, about 2 MHz, about 3 MHz, about 4 MHz, about 5 MHz, about 6 MHz, about 7 MHz, about 8 MHz, about 9 MHz, about 10 MHz, about 11 MHz, about 12 MHz, about 13 MHz, about 14 MHz, about 15 MHz, and the like, as well as a range formed from any of the above values (e.g., a range of from about 50 kHz to about 10 MHz, a range of from about 50 kHz to about 1 MHz, a range of from about 1 MHz to about 10 MHz, a range of from about 50 kHz to about 500 kHz, a range of from about 100 kHz to about 300 kHz, a range of from about 100 kHz to about 200 kHz, a range of from about 150 kHz to about 300 kHz, a range of from about 50 kHz to about 140 kHz, etc.), and a range that combines two integers that fall between two of the above-referenced values (e.g., a range of from about 122 kHz to about 313 kHz, a range of from about 78 kHz to about 298 kHz, etc.).

In certain particular (but non-limiting) embodiments, the alternating electric field may be imposed at two or more different frequencies, either simultaneously or sequentially during treatment (i.e., the frequency may be changed during treatment). When two or more frequencies are present, each frequency is selected from any of the above-referenced values, or a range formed from any of the above-referenced values, or a range that combines two integers that fall between two of the above-referenced values.

The alternating electric field may have any field strength in the intestine of the subject so long as the alternating electric field is capable of increasing permeability of at least a portion of the intestinal epithelia of the subject (such as, but not limited to, by loosening tight junctions between intestinal epithelial cells, delocalizing claudin-2 in intestinal tight junctions, etc.), in accordance with the present disclosure. For example (but not by way of limitation), the alternating electric field may have a field strength of at least about 1 V/cm, about 1.5 V/cm, about 2 V/cm, about 2.5 V/cm, about 3 V/cm, about 3.5 V/cm, about 4 V/cm, about 4.5 V/cm, about 5 V/cm, about 5.5 V/cm, about 6 V/cm, about 6.5 V/cm, about 7 V/cm, about 7.5 V/cm, about 8 V/cm, about 9 V/cm, about 9.5 V/cm, about 10 V/cm, about 10.5 V/cm, about 11 V/cm, about 11.5 V/cm, about 12 V/cm, about 12.5 V/cm, about 13 V/cm, about 13.5 V/cm, about 14 V/cm, about 14.5 V/cm, about 15 V/cm, about 15.5 V/cm, about 16 V/cm, about 16.5 V/cm, about 17 V/cm, about 17.5 V/cm, about 18 V/cm, about 18.5 V/cm, about 19 V/cm, about 19.5 V/cm, about 20 V/cm, and the like, as well as a range formed from any of the above values (e.g., a range of from about 1 V/cm to about 20 V/cm, a range of from about 1 V/cm to about 10 V/cm, a range of from about 1 V/cm to about 4 V/cm, etc.), and a range that combines two integers that fall between two of the above-referenced values (e.g., a range of from about 1.1 V/cm to about 18.6 V/cm, a range of from about 1.2 V/cm to about 9.8 V/cm, a range of from about 1.3 V/cm to about 4.7 V/cm, etc.).

The alternating electric field may be applied for any period of time sufficient to increase the permeability of the intestinal epithelia (such as, but not limited to, by loosening tight junctions between intestinal epithelial cells, delocalizing claudin-2 in intestinal tight junctions, etc.). For example, but not by way of limitation, the alternating electric field may be applied for at least about 1 hour, about 2 hours, about 3 hours, about 4 hours, about 5 hours, about 6 hours, about 7 hours, about 8 hours, about 9 hours, about 10 hours, about 11 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, and the like, as well as a range formed from any of the above values (e.g., a range of from about 24 hours to about 72 hours, etc.), and a range that combines two integers that fall between two of the above-referenced values (e.g., a range of from about 14 hours to about 68 hours, etc.).

The total period of time that the alternating electric field is applied may be achieved in a continuous or intermittent manner. That is, when the alternating electric field is applied for a shorter period of time (such as, but not limited to, less than about 12 or 24 hours), the alternating electric field may be continuously applied over that period of time. However, when the alternating electric field is applied for a longer period of time (such as, but not limited to, a period of about 24 hours or greater), the treatment period may include one or more breaks during the application cycle that separate two or more application sections, whereby the application sections and breaks combine to form the total application period. When breaks are present, the breaks should typically constitute about 20% or less of the treatment time, so that the alternating electric field is applied for at least about 80% or more of the treatment time. For example, but not by way of limitation, the alternating electric field should be applied for at least about 19 hours of each 24-hour period. In addition, the longer that the alternating electric field is applied, the higher the efficacy will be.

In a particular (but non-limiting) embodiment, the alternating electric field may be applied for a limited period of time (such as, but not limited to, about 24 hours to about 72 hours) to increase the permeability of the intestinal epithelia and open the intestinal barrier so as to increase the intestinal permeability of the administered substance (which may be administered after application of the alternating electric field has commenced and before or after application of the alternating electric field has elapsed). However, in contrast to methods of using alternating electric fields for the application of tumor treating fields to the abdomen for treatment of abdominal cancer(s) (such as, but not limited to, ovarian cancer and pancreatic cancer), application of the alternating electric field may then cease after the limited period of time passes, so that the tight junctions can be allowed to close and the permeability of the intestinal epithelia can return to at or near normal levels (thereby limiting adverse effects from the increased permeability, such as (but not limited to) pathogen infiltration, bacterial infection, etc.). Application of the alternating electric field for a limited period of time can then be repeated (for the same or different period of time) before a second dose of the substance is administered (with the dose being administered after the application of the alternating electric field has commenced and before or after application of the alternating electric field has elapsed). The period of time in between the alternating electric field application steps will be related to the dosing schedule for the substance; non-limiting examples of periods of time when no alternating electric field is applied to the subject (i.e., the period between two application steps) include about 12 hours, about 18 hours, about 24 hours, about 48 hours, about 72 hours, about 96 hours, about 1 week, about 2 weeks, about 3 weeks, about 4 weeks, about 1 month, about 2 months, about 3 months, about 4 months, about 5 months, about 6 months, and the like, as well as a range formed from any of the above values (e.g., a range of from about 24 hours to about 1 week, etc.), and a range that combines two integers that fall between two of the above-referenced values (e.g., a range of from about 6 days to about 15 days, etc.).

In certain particular (but non-limiting) embodiments, the desired increased permeability of the intestinal epithelia of the subject is achieved by generating the alternating electric field at a sufficient frequency and field strength and for a sufficient period of time to result in at least about a 10% reduction in claudin-2 expression in at least a portion of the tight junctions of the duodenum of the subject (such as, but not limited to, at least a majority of the tight junctions of the duodenum of the subject). For example (but not by way of limitation), application of the alternating electric field may result in reduction in claudin-2 expression in at least a portion of the tight junctions of the duodenum by at least about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 45%, about 50%, or higher, as well as a range formed from two of the above values (e.g., a range of from about 10% to about 25%, etc.).

Any substance(s) that is known in the art or otherwise contemplated herein for oral administration to a subject and for which increased permeability across the intestinal wall into the bloodstream is desired can be utilized as the at least one substance in the methods of the present disclosure. For example (but not by way of limitation), the at least one substance may be selected from a therapeutic drug, a vitamin, a food supplement, a nutritional supplement, and the like, as well as any combinations thereof. Particular (but non-limiting) examples of therapeutic drugs that can be utilized in accordance with the present disclosure include antibiotics, antiviral medications, antifungal medications, non-steroidal anti-inflammatory drugs (NSAIDS), antihypertensive medications, nanoparticles (used alone or as a carrier for other substances), biologic/immunotherapy drugs, chemotherapeutic drugs (and in particular, but not by way of limitation, chemotherapeutic drugs for non-solid tumor cancers (e.g., hematological cancers like leukemia, lymphoma, myeloma, etc.), cancers that are hard to reach with typical TTFields treatment (e.g., prostate, etc.), and/or metastatic cancer), radiotherapy drugs, and the like, as well as any combinations thereof. Particular (but non-limiting) examples of vitamins that can be utilized in accordance with the present disclosure include vitamins A, B₁, B₂, B₃, B₆, B₁₂, folate, biotin, pantothenate, vitamins C, D, E and K, and the like, as well as any combinations thereof. Particular (but non-limiting) examples of food supplements that can be utilized in accordance with the present disclosure include amino acids, lipids, carbohydrates, and the like, as well as any combinations thereof. Particular (but non-limiting) examples of nutritional supplements that can be utilized in accordance with the present disclosure include minerals (such as, but not limited to, iron, zinc, copper, calcium, and the like, as well as combinations thereof), glucosamine, probiotics, Omega-3 fatty acids, and the like, as well as any combinations thereof. In addition, substances from two or more of the above classes of compounds may be utilized in combination with one another; for example (but not by way of limitation), at least one therapeutic drug may be utilized in combination with at least one vitamin, at least one food supplement, and/or at least one nutritional supplement.

In certain particular (but non-limiting) embodiments, the at least one substance has a size and/or molecular weight that affects its ability to be absorbed quickly, and therefore increased permeability across the intestinal wall into the bloodstream is desired. For example, but not by way of limitation, the substance may have a molecular weight of at least about 5 kDa. Non-limiting examples of molecular weights possessed by substances that could benefit from increased permeability thereof include about 5 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa, about 17 kDa, about 18 kDa, about 19 kDa, about 20 kDa, about 21 kDa, about 22 kDa, about 23 kDa, about 24 kDa, about 25 kDa, about 26 kDa, about 27 kDa, about 28 kDa, about 29 kDa, about 30 kDa, about 31 kDa, about 32 kDa, about 33 kDa, about 34 kDa, about 35 kDa, about 36 kDa, about 37 kDa, about 38 kDa, about 39 kDa, about 40 kDa, about 41 kDa, about 42 kDa, about 43 kDa, about 44 kDa, about 45 kDa, about 46 kDa, about 47 kDa, about 48 kDa, about 49 kDa, about 50 kDa, and higher, as well as a range formed from two of the above values (e.g., a range of from about 5 kDa to about 40 kDa, etc.).

In certain particular (but non-limiting) embodiments, the at least one substance is orally administered after the application of the alternating electric field has begun. In particular (but not by way of limitation), the at least one substance may be orally administered during application of the alternating electric field and/or after application of the alternating electric field has elapsed.

For example (but not by way of limitation), the at least one substance may be orally administered after application of the alternating electric field has commenced by a period of at least about 3 hours, about 6 hours, about 9 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, and the like, as well as a range formed from any of the above values (e.g., a range of from about 24 hours to about 96 hours, etc.), and a range that combines two integers that fall between two of the above-referenced values (e.g., a range of from about 14 hours to about 94 hours, etc.). In a particular (but non-limiting) embodiment, the at least one substance is orally administered at least about 24 hours after application of the alternating electric field has begun.

In other non-limiting examples, the at least one substance may be orally administered after the period of time that the alternating electric field is applied has elapsed, wherein the at least one substance is administered within about 3 hours, about 6 hours, about 9 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, and the like, of when the period of time elapsed. In a particular (but non-limiting) embodiment, the at least one substance is orally administered within about 96 hours of when the period of time elapsed.

In certain particular (but non-limiting) embodiments, the method includes one or more additional steps. For example (but not by way of limitation), the method may further include the step of (3) discontinuing the application of the alternating electric field (such as, but not limited to) to allow the intestinal epithelia to recover (i.e., to regain a level of impermeability, such as returning to or near the level of impermeability before onset of treatment and thereby maintaining the intestinal boundaries from possible pathogen infiltration). In addition, any of steps (1), (2), and/or (3) may be repeated one or more times.

In certain particular (but non-limiting) embodiments, the method involves concurrent therapy with two or more substances. As such, the method may include an additional step of (4) administering at least a second substance to the subject, wherein the increased permeability of the intestinal epithelia allows both the first and second substances to cross the intestinal wall into the bloodstream. In a particular (but non-limiting) embodiment, the at least second substance may be selected from any of the substances disclosed or otherwise contemplated herein for use as the first substance (e.g., a therapeutic drug, a vitamin, a food supplement, and/or a nutritional supplement, and the like).

When present, step (4) may be performed simultaneously or wholly or partially sequentially with the administration of the first substance in step (2). In addition, this optional additional administration step may be performed before or after the application of the alternating electric field has begun, and during application of the alternating electric field and/or after application of the alternating electric field has elapsed, in the same manner(s) and time frame(s) as described above for the first substance.

That is, for example (but not by way of limitation), the second substance may be orally administered after application of the alternating electric field has commenced by a period of at least about 3 hours, about 6 hours, about 9 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, and the like, as well as a range formed from any of the above values (e.g., a range of from about 24 hours to about 96 hours, etc.), and a range that combines two integers that fall between two of the above-referenced values (e.g., a range of from about 14 hours to about 94 hours, etc.). In a particular (but non-limiting) embodiment, the second substance is orally administered at least about 24 hours after application of the alternating electric field has begun.

In other non-limiting examples, the second substance may be orally administered after the period of time that the alternating electric field is applied has elapsed, wherein the second substance is administered within about 3 hours, about 6 hours, about 9 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, and the like, of when the period of time elapsed. In a particular (but non-limiting) embodiment, the second substance is orally administered within about 96 hours of when the period of time elapsed.

In addition, for example (but not by way of limitation), the second substance may be orally administered after administration of the first substance by a period of at least about 1 minute, about 5 minutes, about 10 minutes, about 15 minutes, about 30 minutes, about 45 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, and the like, as well as a range formed from any of the above values (e.g., a range of from about 24 hours to about 96 hours, etc.), and a range that combines two integers that fall between two of the above-referenced values (e.g., a range of from about 14 hours to about 94 hours, etc.). In a particular (but non-limiting) embodiment, the second substance is orally administered at least about 12 hours after administration of the first substance.

Any of steps (1) and (2) and optional steps (3) and/or (4) may be repeated one or more times. Each of the steps can be repeated as many times as necessary. When steps (1) and (3) are repeated, the transducer arrays may be placed in slightly different positions on the subject's abdomen than their original placement; relocation of the arrays in this manner may further increase the permeability of the intestinal epithelia. In addition, the steps (2) and (4) of administering substances may be repeated various times and at various intervals to follow any known and/or generally accepted dosage regimen for the substance.

In addition, the methods of the present disclosure include performing step (4) in the absence of step (3), as well as performing step (3) in the absence of step (4). That is, the scope of the methods disclosed herein includes performing steps (1)-(2) (as well as repeating each step as many times as necessary), performing steps (1)-(3) (as well as repeating each step as many times as necessary), performing steps (1)-(2) and (4) (as well as repeating each step as many times as necessary), and performing steps (1)-(4) (as well as repeating each step as many times as necessary).

While the use of concurrent therapy with two substances is explicitly described above, it will be understood that the scope of the present disclosure further includes concurrent therapy with three or more substances. As such, the method can include one or more additional steps of administering an additional substance to the subject (similar to steps (2) and (4)). Any additional substances administered in the method may be selected from any of the substances disclosed or otherwise contemplated herein for use as the first or second substances (e.g., a therapeutic drug, a vitamin, a food supplement, and/or a nutritional supplement, and the like); in addition, administration of any additional substances can be performed simultaneously or wholly or partially sequentially with the administration of the first and/or second substance and in the same manner(s) and time frame(s) as described above for the first and second substances.

Any subjects that would benefit from increased absorption of at least one ingested substance into their bloodstream may be treated in accordance with the methods of the present disclosure. In certain particular (but non-limiting) embodiments, the subject is tumor-free, is free of any solid cancers, is free of an abdominal cancer, has not been diagnosed with cancer, and/or has not been diagnosed with an abdominal cancer (e.g., pancreatic or ovarian)). In addition, in certain particular (but non-limiting) embodiments, the subject may have (or be predisposed to) a malabsorption condition.

Certain non-limiting embodiments of the present disclosure are directed to a method of treating or reducing the occurrence of a malabsorption condition in a subject. In this method, any of the alternating electric field(s) disclosed or otherwise contemplated herein is applied to at least a portion of the subject's abdomen for a period of time. As described above, application of the alternating electric field increases permeability of at least a portion of an intestinal epithelia of the subject. The alternating electric field may be applied at any of the frequencies and field strengths and for any periods of time disclosed or otherwise contemplated herein.

Any malabsorption condition that can be alleviated, lessened, or the occurrence reduced upon increasing the permeability of the intestinal epithelia can be treated by the methods of the present disclosure. Non-limiting examples of malabsorption conditions that can be treated (or for which the occurrence can be reduced) by the methods of the present disclosure include an intestinal resection, inflammatory bowel disease, cystic fibrosis, chronic pancreatitis, lactose intolerance, Celiac disease, Whipple disease, Schwachman-Diamond syndrome, cow's milk protein intolerance (CMPI), milk soy protein intolerance (MSPI), biliary atresia, abetalipoproteinemia (ABL), juvenile pernicious anemia, a restrictive and/or malabsorptive bariatric surgery (such as, but not limited to, gastric banding, gastric bypass, gastric stapling, sleeve gastrectomy, duodenal switch, and the like), a parasitic infection, and combinations thereof. Non-limiting examples of parasitic infections that can be treated include infections caused by any of the parasites Diphyllobothrium latum, Giardia lamblia, Strongyloides stercoralis, Necator americanus, and combinations thereof.

The methods of treating a malabsorption condition disclosed herein may be utilized simply to increase absorption of nutrients from ingested food, and therefore may not require the administration of any additional substances. However, in certain particular (but non-limiting) embodiments, the method of treating or reducing the occurrence of a malabsorption condition in a subject may further include the step of orally administering at least one of any of the substances described herein above or otherwise contemplated herein to the subject, wherein the increased permeability of the intestinal epithelia allows the at least one substance to cross an intestinal wall into the bloodstream at an increased rate compared to the amount of substance that crosses the intestinal wall in the absence of the alternating electric field. This administration step may be performed before or after the application of the alternating electric field has begun, and during application of the alternating electric field and/or after application of the alternating electric field has elapsed, in the same manner(s) and time frame(s) as described above.

Certain non-limiting embodiments of the present disclosure are related to kits that include any of the components of the TTField generating systems (such as, but not limited to, one or more transducer arrays and/or one or more hydrogel compositions, as disclosed in U.S. Pat. Nos. 7,016,725; 7,089,054; 7,333,852; 7,565,205; 8,244,345; 8,715,203; 8,764,675; 10,188,851; and 11,452,863; and in US Patent Application Nos. US 2018/0001078; US 2018/0160933; US 2019/0117956; US 2019/0307781; and US 2019/0308016) and one or more of any of the substances disclosed or otherwise contemplated herein.

In a particular (but non-limiting) embodiment, the kit may further include instructions for performing any of the methods disclosed or otherwise contemplated herein. For example (but not by way of limitation), the kit may include instructions for applying one or more components of an alternating electric field generating system to the skin of the patient's abdomen, instructions for applying the alternating electric field to the patient's abdomen, instructions for when and how to administer the one or more substances, and/or instructions for when to activate and turn off the alternating electric field in relation to the administration of the one or more substances.

In addition to the components described in detail herein above, the kits may further contain other component(s)/reagent(s) for performing any of the particular methods described or otherwise contemplated herein. For example (but not by way of limitation), the kits may additionally include: (i) components for preparing the skin prior to disposal of the hydrogel compositions and/or transducer arrays thereon (e.g., a razor, a cleansing composition or wipe/towel, etc.); (ii) components for removal of the gel/transducer array(s); and/or (iii) components for cleansing of the skin after removal of the gel/transducer array(s). The nature of these additional component(s)/reagent(s) will depend upon the particular treatment format, and identification thereof is well within the skill of one of ordinary skill in the art; therefore, no further description thereof is deemed necessary. Also, the components/reagents present in the kits may each be in separate containers/compartments, or various components/reagents can be combined in one or more containers/compartments, depending on the sterility, cross-reactivity, and stability of the components/reagents.

The kit may be disposed in any packaging that allows the components present therein to function in accordance with the present disclosure. In certain non-limiting embodiments, the kit further comprises a sealed packaging in which the components are disposed. In certain particular (but non-limiting) embodiments, the sealed packaging is substantially impermeable to air and/or substantially impermeable to light.

In addition, the kit can further include a set of written instructions explaining how to use one or more components of the kit. A kit of this nature can be used in any of the methods described or otherwise contemplated herein.

In certain non-limiting embodiments, the kit has a shelf life of at least about six months, such as (but not limited to), at least about nine months, or at least about 12 months.

### EXAMPLE

An Example is provided hereinbelow. However, the present disclosure is to be understood to not be limited in its application to the specific experimentation, results, and laboratory procedures disclosed herein after. Rather, the Example is simply provided as one of various embodiments and is meant to be exemplary, not exhaustive.

As stated above in the Background section, absorption of orally administered drugs is mostly done in the small intestine, but various physiological barriers exist that limit the amount of ingested substance that is absorbed through the intestine and into the bloodstream. In particular, modulation of tight junction permeability has often been attempted as a means to increase the ability of therapeutic substances to cross the epithelium.

It is known that claudin-2 is expressed in the duodenum and colon, and that dysregulation of claudin proteins has been observed in inflammatory states associated with a leaky epithelial barrier. In particular, reduction in claudin-2 expression has been associated with the weakening of epithelial tight junctions (TJs) and increasing the permeability of the intestinal epithelia.

In this Example, the effects of TTFields on the permeability of intestinal epithelia were examined. The expression of claudin-2 in the colon and duodenum was selected for this analysis, based on the teachings of Dan et al. (J Biol Chem (2019) 294(42):15446-15465), Zhu et al. (Frontiers in Immunology (2019) 10:1441), and Xiangfe et al. (Journal of Mater Chem B (2019) 7:6310-6320).

Male and female rats were treated with TTFields (150 kHz) via a torso array for two weeks, and then colon and duodenum tissue samples were taken from the treated rats (as well as heat (i.e., sham control) rats) and processed via paraffin-embedding. The rats tested were healthy and did not bear any tumors.

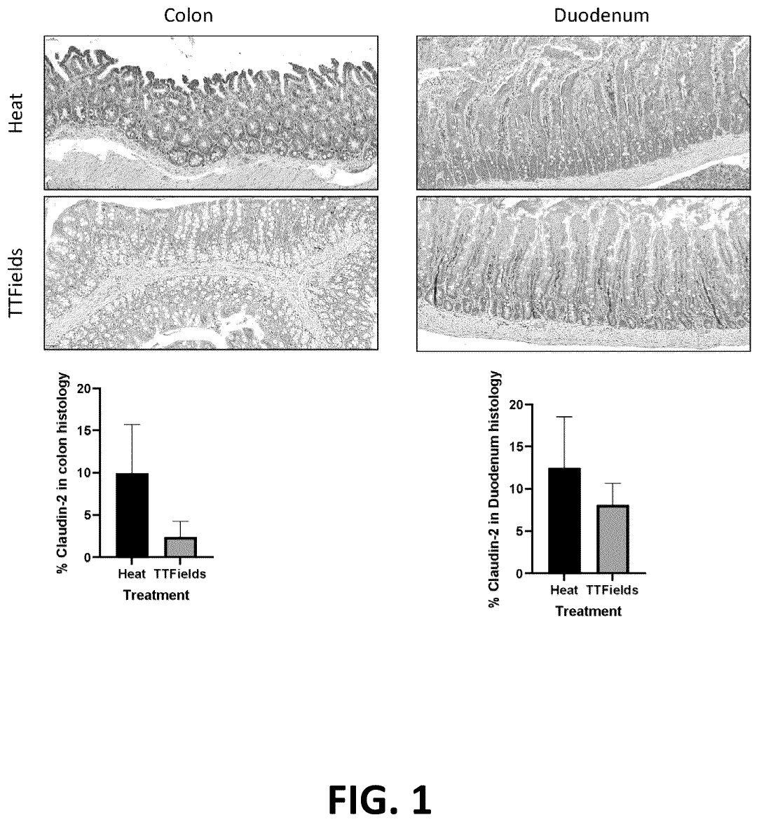

The experimental groups included: (i) heat (i.e., sham control) colon, (ii) heat duodenum, (iii) TTFields colon, and (iv) TTFields duodenum. Four paraffin samples from each experimental group were sectioned and stained for claudin-2 (Cat E-AB-17706, Elabscience, Houston, TX) by immunohistochemistry (FIG. 1, top panel). The slides were scanned, and at least three images were taken from each slide and quantified by ImageJ software (FIG. 1, lower panel). As can be seen in FIG. 1, the application of TTFields substantially decreased claudin-2 expression in the colon as well as in the duodenum.

Therefore, this Example demonstrates that the application of TTFields increases the permeability of the intestinal epithelia and thereby increases the ability of an orally administered substance to cross the intestinal wall into the bloodstream.

Without being bound by the theory, it is believed that the increased permeability arises from the application of alternating electric fields, which causes a reduction in the expression of claudin-2, and claudin-2 reduction in the duodenum and colon is associated with the weakening of epithelial tight junctions (TJs) and increasing the permeability of the intestinal epithelia.

### Non-Limiting Illustrative Embodiments of the Inventive Concept(s)

Illustrative embodiment 1. A method of increasing absorption of at least one ingested substance into a bloodstream of a subject, the method comprising the steps of: (1) applying an alternating electric field to at least a portion of an abdomen of the subject for a period of time, wherein application of the alternating electric field increases permeability of at least a portion of an intestinal epithelia of the subject; and (2) orally administering the at least one substance to the subject, wherein the increased permeability of the intestinal epithelia allows the at least one substance to cross an intestinal wall into the bloodstream.

Illustrative embodiment 2. The method of illustrative embodiment 1, wherein the at least one substance is orally administered after the application of the alternating electric field has begun.

Illustrative embodiment 3. The method of illustrative embodiment 2, wherein the at least one substance is orally administered at least 24 hours after application of the alternating electric field has begun.

Illustrative embodiment 4. The method of any one of illustrative embodiments 1-3, wherein the period of time that the alternating electric field is applied is in a range of from about 24 hours to about 72 hours.

Illustrative embodiment 5. The method of any one of illustrative embodiments 1-4, wherein the at least one substance is orally administered after the period of time has elapsed.

Illustrative embodiment 6. The method of illustrative embodiment 5, wherein the at least one substance is orally administered within about 96 hours of when the period of time elapsed.

Illustrative embodiment 7. The method of any one of illustrative embodiments 1-4, wherein the at least one substance is orally administered before the period of time the alternating electric field is applied has elapsed.

Illustrative embodiment 8. The method of any one of illustrative embodiments 1-7, wherein the alternating electric field is applied at a frequency in a range of from about 50 kHz to about 10 MHz.

Illustrative embodiment 9. The method of illustrative embodiment 8, wherein the alternating electric field is applied at a frequency in a range of from about 50 kHz to about 140 kHz.

Illustrative embodiment 10. The method of any one of illustrative embodiments 1-9, wherein the alternating electric field is applied at a frequency in a range of from about 1 MHz to about 10 MHz.

Illustrative embodiment 11. The method of any one of illustrative embodiments 1-10, wherein the alternating electric field has a field strength of at least 1 V/cm.

Illustrative embodiment 12. The method of any one of illustrative embodiments 1-11, further comprising the step of discontinuing the application of the alternating electric field to allow the intestinal epithelia to recover.

Illustrative embodiment 13. The method of any one of illustrative embodiments 1-12, wherein steps (1) and (2) are repeated one or more times.

Illustrative embodiment 14. The method of illustrative embodiment 13, wherein the method further includes ceasing application of the alternating electric field for a period of time in a range of from about 24 hours to about 1 month between repetitions of step (1).

Illustrative embodiment 15. The method of any one of illustrative embodiments 1-14, wherein the at least one substance is selected from the group consisting of a therapeutic drug, a vitamin, a food supplement, a nutritional supplement, and combinations thereof.

Illustrative embodiment 16. The method of illustrative embodiment 15, wherein the at least one substance is selected from the group consisting of an antibiotic, an anti-viral medication, a nanoparticle, a food supplement (e.g., amino acids, lipids, and/or carbohydrates), a nutritional supplement (e.g., iron, folic acid, and/or vitamin B12), an antihypertensive medication, a non-steroidal anti-inflammatory drug (NSAID), and combinations thereof.

Illustrative embodiment 17. A method of treating or reducing the occurrence of a malabsorption condition in a subject, the method comprising the steps of: applying an alternating electric field to at least a portion of an abdomen of the subject for a period of time, wherein application of the alternating electric field increases permeability of at least a portion of an intestinal epithelia of the subject.

Illustrative embodiment 18. The method of illustrative embodiment 17, wherein the malabsorption condition is selected from the group consisting of an intestinal resection, inflammatory bowel disease, cystic fibrosis, chronic pancreatitis, lactose intolerance, Celiac disease, Whipple disease, Schwachman-Diamond syndrome, cow's milk protein intolerance (CMPI), milk soy protein intolerance (MSPI), biliary atresia, abetalipoproteinemia (ABL), juvenile pernicious anemia, a restrictive and/or malabsorptive bariatric surgery, a parasitic infection, and combinations thereof.

Illustrative embodiment 19. The method of illustrative embodiment 18, wherein the parasitic infection is caused by a parasite selected from the group consisting of Diphyllobothrium latum, Giardia lamblia, Strongyloides stercoralis, Necator americanus, and combinations thereof.

Illustrative embodiment 20. The method of any one of illustrative embodiments 17-19, wherein the alternating electric field is applied at a frequency in a range of from about 50 kHz to about 10 MHz.

Illustrative embodiment 21. The method of illustrative embodiment 20, wherein the alternating electric field is applied at a frequency in a range of from about 1 MHz to about 10 MHz.

Illustrative embodiment 22. The method of illustrative embodiment 20, wherein the alternating electric field is applied at a frequency in a range of from about 50 kHz to about 140 kHz.

Illustrative embodiment 23. The method of any one of illustrative embodiments 17-22, wherein the alternating electric field has a field strength of at least 1 V/cm.

Illustrative embodiment 24. The method of any one of illustrative embodiments 17-23, wherein the period of time that the alternating electric field is applied is in a range of from about 24 hours to about 72 hours.

Illustrative embodiment 25. The method of any one of illustrative embodiments 17-24, wherein the method includes repeating the application of the alternating electric field one or more times, and wherein the method further includes ceasing application of the alternating electric field for a period of time in a range of from about 24 hours to about 1 month between repetitions of the application step.

Illustrative embodiment 26. The method of any one of illustrative embodiments 17-25, further comprising the step of orally administering at least one substance to the subject, wherein the increased permeability of the intestinal epithelia allows the at least one substance to cross an intestinal wall into the bloodstream.

Illustrative embodiment 27. The method of illustrative embodiment 26, wherein the at least one substance is selected from the group consisting of a therapeutic drug, a vitamin, a food supplement, a nutritional supplement, and combinations thereof.

Illustrative embodiment 28. The method of illustrative embodiment 27, wherein the at least one substance is selected from the group consisting of an antibiotic, an anti-viral medication, a nanoparticle, a food supplement (e.g., amino acids, lipids, and/or carbohydrates), a nutritional supplement (e.g., iron, folic acid, and/or vitamin B12), an antihypertensive medication, a non-steroidal anti-inflammatory drug (NSAID), and combinations thereof.

Illustrative embodiment 29. The method of any one of illustrative embodiments 26-28, wherein the at least one substance is orally administered after the application of the alternating electric field has begun.

Illustrative embodiment 30. The method of any one of illustrative embodiments 26-29, wherein the at least one substance is orally administered after the period of time that the alternating electric field is applied has elapsed.

Illustrative embodiment 31. The method of any one of illustrative embodiments 1-30, wherein the subject has not been diagnosed with cancer.

Illustrative embodiment 32. The method of any one of illustrative embodiments 1-31, wherein the subject has not been diagnosed with an abdominal cancer.

Illustrative embodiment 33. The method of any one of illustrative embodiments 1-32, wherein the subject is free of any solid cancers.

Illustrative embodiment 34. The method of any one of illustrative embodiments 1-33, wherein the subject is free of an abdominal cancer.

Illustrative embodiment 35. The method of any one of illustrative embodiments 1-34, wherein the alternating electric field is applied at a sufficient frequency and field strength and for a sufficient period of time to reduce claudin-2 expression in at least a portion of the tight junctions of the duodenum of the subject by at least about 10%.

Illustrative embodiment 36. The method of any one of illustrative embodiments 1-35, wherein the at least one substance has a molecular weight of at least about 5 kDa.

Illustrative embodiment 37. The method of any one of illustrative embodiments 1-36, wherein the at least one substance has a molecular weight in a range of from about 5 kDa to about 40 kDa.

Illustrative embodiment 38. At least one substance having a molecular weight in a range of from about 5 kDa to about 40 kDa for use in any of the methods of illustrative embodiments 1-37.

Illustrative embodiment 39. Use of at least one substance having a molecular weight in a range of from about 5 kDa to about 40 kDa in any of the methods of illustrative embodiments 1-37.

Illustrative embodiment 40. At least one substance for use in any of the methods of illustrative embodiments 1-37, wherein the at least one substance is selected from the group consisting of a therapeutic drug, a vitamin, a food supplement, a nutritional supplement, and combinations thereof.

Illustrative embodiment 41. The at least one substance of illustrative embodiment 40, wherein the at least one substance is selected from the group consisting of an antibiotic, an anti-viral medication, a nanoparticle, a food supplement (e.g., amino acids, lipids, and/or carbohydrates), a nutritional supplement (e.g., iron, folic acid, and/or vitamin B12), an antihypertensive medication, a non-steroidal anti-inflammatory drug (NSAID), and combinations thereof.

Illustrative embodiment 42. Use of at least one substance in any of the methods of illustrative embodiments 1-37, wherein the at least one substance is selected from the group consisting of a therapeutic drug, a vitamin, a food supplement, a nutritional supplement, and combinations thereof.

Illustrative embodiment 43. The at least one substance of illustrative embodiment 42, wherein the at least one substance is selected from the group consisting of an antibiotic, an anti-viral medication, a nanoparticle, a food supplement (e.g., amino acids, lipids, and/or carbohydrates), a nutritional supplement (e.g., iron, folic acid, and/or vitamin B12), an antihypertensive medication, a non-steroidal anti-inflammatory drug (NSAID), and combinations thereof.

While the attached disclosures describe the inventive concept(s) in conjunction with the specific experimentation, results, and language set forth hereinafter, it is evident that many alternatives, modifications, and variations will be apparent to those skilled in the art. Accordingly, it is intended to embrace all such alternatives, modifications, and variations that fall within the spirit and broad scope of the present disclosure.

What is claimed is:

1. A method of increasing absorption of at least one ingested substance into a bloodstream of a subject, the method comprising the steps of:
(1) applying an alternating electric field to at least a portion of an abdomen of the subject for a period of time, wherein application of the alternating electric field increases permeability of at least a portion of an intestinal epithelia of the subject; and
(2) orally administering the at least one substance to the subject after application of the alternating electric field has begun, wherein the increased permeability of the intestinal epithelia allows the at least one substance to cross an intestinal wall into the bloodstream.

2. The method of claim 1, wherein the subject has not been diagnosed with an abdominal cancer.

3. The method of claim 1, wherein the alternating electric field is applied at a sufficient frequency and for a sufficient period of time to reduce claudin-2 expression in at least a portion of the tight junctions of the duodenum of the subject by at least about 10%.

4. The method of claim 1, wherein the at least one substance has a molecular weight in a range of from about 5 kDa to about 40 kDa.

5. The method of claim 1, wherein the at least one substance is orally administered within about 96 hours of when the period of time elapsed.

6. The method of claim 1, wherein the alternating electric field is applied at a frequency in a range of from about 50 kHz to about 10 MHz and for a period of time in a range of from about 24 hours to about 72 hours.

7. The method of claim 1, further comprising the step of discontinuing the application of the alternating electric field to allow the intestinal epithelia to recover.

8. The method of claim 1, wherein steps (1) and (2) are repeated one or more times, and wherein the method further includes ceasing application of the alternating electric field for a period of time in a range of from about 24 hours to about 1 month between repetitions of step (1).

9. The method of claim 1, wherein the at least one substance is selected from the group consisting of a therapeutic drug, a vitamin, a food supplement, a nutritional supplement, and combinations thereof.

10. A method of treating or reducing the occurrence of a malabsorption condition in a subject, the method comprising the steps of:
applying an alternating electric field to at least a portion of an abdomen of the subject for a period of time, wherein application of the alternating electric field increases permeability of at least a portion of an intestinal epithelia of the subject.

11. The method of claim 10, wherein the malabsorption condition is selected from the group consisting of an intestinal resection, inflammatory bowel disease, cystic fibrosis, chronic pancreatitis, lactose intolerance, Celiac disease, Whipple disease, Schwachman-Diamond syndrome, cow's milk protein intolerance (CMPI), milk soy protein intolerance (MSPI), biliary atresia, abetalipoproteinemia (ABL), juvenile pernicious anemia, a restrictive and/or malabsorptive bariatric surgery, a parasitic infection, and combinations thereof.

12. The method of claim 11, wherein the parasitic infection is caused by a parasite selected from the group consisting of Diphyllobothrium latum, Giardia lamblia, Strongyloides stercoralis, Necator americanus, and combinations thereof.

13. The method of claim 10, wherein the subject has not been diagnosed with an abdominal cancer.

14. The method of claim 10, wherein the alternating electric field is applied at a sufficient frequency and for a sufficient period of time to reduce claudin-2 expression in at least a portion of the tight junctions of the duodenum of the subject by at least about 10%.

15. The method of claim 10, wherein the alternating electric field is applied at a frequency in a range of from about 50 kHz to about 10 MHz and for a period of time in a range of from about 24 hours to about 72 hours.

16. The method of claim 10, wherein the step of applying the alternating electric field is repeated one or more times, and wherein the method further includes ceasing application of the alternating electric field for a period of time in a range of from about 24 hours to about 1 month between repetitions of the application step.

17. The method of claim 10, further comprising the step of orally administering at least one substance to the subject after the application of the alternating electric field has begun, wherein the increased permeability of the intestinal epithelia allows the at least one substance to cross an intestinal wall into the bloodstream.

18. The method of claim 17, wherein the at least one substance is selected from the group consisting of a therapeutic drug, a vitamin, a food supplement, a nutritional supplement, and combinations thereof.

19. The method of claim 17, wherein the at least one substance is orally administered.

20. The method of claim 17, wherein the at least one substance has a molecular weight in a range of from about 5 kDa to about 40 kDa.
